(19) United States
(12) Patent Application Publication
(10) Pub. No.: US 2004/0009168 A1
(43) Pub. Date: Jan. 15, 2004

(54) Multidose antibody formulation

(76) Inventors: Elizabet Kaisheva, Belmont, CA (US); Supriya Gupta, Sunnyvale, CA (US)

Correspondence Address: HOWREY SIMON ARNOLD & WHITE, LLP, BOX 34, 301 RAVENSWOOD AVE., MENLO PARK, CA, 94025, US

(21) Appl. No.: 10/409,477

(22) Filed: Apr. 7, 2003

Related U.S. Application Data
(60) Provisional application No. 60/370,660, filed on Apr. 5, 2002.

(51) Int. Cl. A61K 39/395

(52) U.S. Cl. 424/141.1; 424/144.1

(57) ABSTRACT

This invention is directed to a multidose pharmaceutical formulation comprising an antibody with one or more preservatives. This formulation is effective in inhibiting the growth of microorganisms. This formulation further retains the physical, chemical, and biological stability of the antibody molecule. In one embodiment of the invention, the pharmaceutical formulation comprises an IgG antibody, 0.15-0.2% (w/v) chlorobutanol, and 0.3-0.5% (w/v) benzyl alcohol. In another embodiment of the invention, the pharmaceutical formulation comprises an IgG antibody, 0.1-0.2% (w/v) chlorobutanol, and 0.05-0.1% (w/v) methyl paraben. In yet another embodiment of the invention, the pharmaceutical formulation comprises an IgG antibody and 0.5-0.75% benzyl alcohol.

This application claims the benefit of U.S. Provisional Application No. 60/370,660 filed Apr. 5, 2002.

### FIELD OF THE INVENTION

The present invention relates generally to the field of pharmaceutical formulation of antibodies. Specifically, the present invention relates to a stable, liquid antibody formulation that is free from antimicrobial activity.

### BACKGROUND OF THE INVENTION

Biopharmaceuticals are often formulated as multidose products because they are convenient for patient administration; they minimize sample wastage when dosage requirements are not ascertained, and they provide dosage flexibility for future drug indications. However, multidose products are easily contaminated by microorganisms. Multidose formulations in general contain antimicrobial agents to protect them from microbial contamination during multiple dosage withdrawals from the vial.

Over the last decade, there has been a significant increase in the number of commercial protein products, but few have been marketed in a multidose configuration (1). The literature on the development of multidose formulations for proteins is also not extensive. The paucity of multidose protein products is due in part to the difficulty of selecting appropriate preservatives.

Selection of optimal preservatives is dependent on a number of factors (1-3). Preservatives that inhibited the growth of microorganisms need to be compatible with the route of administration and be effective against various strains of fungi and bacteria (2, 3). Preservative activity is pH-specific (eg, benzyl alcohol is effective only in the pH range of 4-7), and thus, the pH of the formulation limits the use of a number of preservatives. Other formulation components impose additional restrictions, for example nonionic surfactants such as the Tweens® inactivate parabens and phenolic preservatives (3). Poor aqueous solubility and concentration loss due to adsorption by rubber stoppers are other concerns in ensuring the long-term antimicrobial efficacy of preservatives (4-6). Acceptance of preservatives in target markets is also important. Many preservatives approved for parenteral use in the US are not approved in Europe and Japan (2). Antimicrobial preservatives are also considerably toxic, and thus the target population's sensitivity to them needs to be carefully evaluated. For example, benzalkonium chloride and EDTA from nebulized solutions have been reported to induce dose-related bronchoconstriction in asthmatics (7).

The effect of preservatives on protein stability is a major concern. Antimicrobial preservatives are known to interact with proteins and cause stability problems such as aggregation (8-10). The effect of preservatives on antibody stability is even a more difficult issue because antibody molecules are very large proteins (150 KD) and have very specific antigen-binding properties. To maintain the activities of antibody molecules, it is very important that the tertiary and quaternary structures of the antibody molecules are not affected by any degradation mechanism.

Therefore, it is challenging to identify formulation-compatible preservatives at concentrations that provide a desired antimicrobial efficacy but do not adversely affect the antibody stability. In addition, regulatory requirements assert that the antimicrobial efficacy of the formulation must satisfy the preservative efficacy test (PET) requirements of the target markets. The PET requirements of the United States Pharmacopoeia (USP) and the European/British Pharmacopoeia (EP/BP) differ considerably, imposing additional constraints in developing multidose formulations (2). Furthermore, in certain cases, preservative such as benzyl alcohol can catalyze protein oxidation, which may also cause protein conformational changes.

The present invention identifies a pharmaceutical antibody formation that is stable, free of microorganism contamination, and suitable for multidose administration.

### BRIEF DESCRIPTION OF FIGURES

FIG. 1 shows response surface plots, which indicate the bacterial and fungal count as a function of the concentration of two preservatives. Counts were measured after 14 days of incubation at room temperature. FIG. 1(a) shows the effect of benzyl alcohol and chlorobutanol on the bacterial count. FIG. 1(b) shows the effect of benzyl alcohol and chlorobutanol on the fungal count. FIG. 1(c) shows the effect of chlorobutanol and methyl paraben on the bacterial count. FIG. 1(d) shows the effect of chlorobutanol and methyl paraben on the fungal count.

### SUMMARY OF THE INVENTION

This invention is directed to a multidose pharmaceutical formulation comprising an antibody with one or more preservatives. This formulation is effective on inhibiting the growth of microorganisms. This formulation further retains the physical, chemical, and biological stability of the antibody molecule.

In one embodiment of the invention, the pharmaceutical formulation comprises an IgG antibody, 0.15-0.2% (w/v) chlorobutanol, and 0.3-0.5% (w/v) benzyl alcohol.

In another embodiment of the invention, the pharmaceutical formulation comprises an IgG antibody, 0.1-0.2% (w/v) chlorobutanol, and 0.05-0.1% (w/v) methyl paraben.

In yet another embodiment of the invention, the pharmaceutical formulation comprises an IgG antibody and 0.5-0.75% benzyl alcohol.

### DETAILED DESCRIPTION OF THE INVENTION

I. Definition

The term “pharmaceutical formulation” refers to preparations which are in such form as to permit the biological activity of the active ingredients to be unequivocally effective, and which contain no additional components which are toxic to the subjects to which the formulation would be administered.

A “stable” formulation is one in which the protein therein essentially retains its physical stability, chemical stability, and biological activity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10:29-90 (1993). Stability can be measured at a selected temperature for a selected time period.

A “stable” liquid antibody formulation is a liquid antibody formulation with no significant changes observed at a refrigerated temperature (2-8° C.) for at least 12 months, preferably 2 years, and more preferably 3 years; or at room temperature (23-27° C.) for at least 3 months, preferably 6 months, and more preferably 1 year. The criteria for stability are as follows. No more than 10%, preferably 5%, of antibody monomer is degraded as measured by SEC-HPLC. The solution is colorless, or clear to slightly opalescent by visual analysis. The concentration, pH and osmolality of the formulation have no more than +/−10% change. Potency is within 70-130%, preferably 80-120% of the control. No more than 10%, preferably 5% of clipping (hydrolysis) is observed. No more than 10%, preferably 5% of aggregation is formed.

An antibody “retains its physical stability” in a pharmaceutical formulation if it shows no significant increase of aggregation, precipitation and/or denaturation upon visual examination of color and/or clarity, or as measured by UV light scattering, size exclusion chromatography (SEC-HPLC) and dynamic light scattering. In addition the protein conformation is not altered. The changes of protein conformation can be evaluated by fluorescence spectroscopy, which determines the protein tertiary structure, and by FTIR spectroscopy, which determines the protein secondary structure.

An antibody “retains its chemical stability” in a pharmaceutical formulation, if it shows no significant chemical alteration. Chemical stability can be assessed by detecting and quantifying chemically altered forms of the protein. Degradation processes that often alter the protein chemical structure include hydrolysis or clipping (evaluated by methods such as size exclusion chromatography and SDS-PAGE), oxidation (evaluated by methods such as by peptide mapping in conjunction with mass spectroscopy or MALDI/TOF/MS), deamidation (evaluated by methods such as ion-exchange chromatography, capillary isoelectric focusing, peptide mapping, isoaspartic acid measurement), and isomerization (evaluated by measuring the isoaspartic acid content, peptide mapping, etc.).

An antibody “retains its biological activity” in a pharmaceutical formulation, if the biological activity of the antibody at a given time is within a predetermined range of the biological activity exhibited at the time the pharmaceutical formulation was prepared. The biological activity of an antibody can be determined, for example, by an antigen binding ELISA assay.

II. Analytical Methods

The following criteria are important in developing a stable pharmaceutical antibody formulation. The antibody formulation contains pharmaceutically acceptable excipients. The antibody formulation is formulated such that the antibody retains its physical, chemical and biological activity. The formulation is preferably stable for at least 1 year at refrigerated temperature (2-8° C.) and 6 months at room temperature (23-27° C.).

The analytical methods for evaluating the product stability include size exclusion chromatography (SEC-HPLC), dynamic light scattering test (DLS), differential scanning calorimetery (DSC), iso-asp quantification, potency, UV at 340 nm, and UV spectroscopy. SEC (J. Pharm. Scien., 83:1645-1650, (1994); Pharm. Res., 11:485 (1994); J. Pharm. Bio. Anal., 15:1928 (1997); J Pharm. Bio. Anal., 14:1133-1140 (1986)) measures percent monomer in the product and gives information of the amount of soluble aggregates and clips. DSC (Pharm. Res., 15:200 (1998); Pharm. Res., 9:109 (1982)) gives information of protein denaturation temperature and glass transition temperature. DLS (American Lab., November (1991)) measures mean diffusion coefficient, and gives information of the amount of soluble and insoluble aggregates. UV at 340 nm measures scattered light intensity at 340 nm and gives information about the amounts of soluble and insoluble aggregates. UV spectroscopy measures absorbance at 278 nm and gives information of protein concentration.

The potency or bioactivity of an antibody can be measured by its ability to bind to its antigen. The specific binding of an antibody to its antigen can be quantitated by any method known to those skilled in the art, for example, an immunoassay, such as ELISA (enzyme-linked immunosorbant assay).

III. Preparation of Antibody

The invention herein relates to a stable aqueous formulation comprising an antibody. The antibody in the formulation is prepared using techniques available in the art for generating antibodies, exemplary methods of which are described in more detail in the following sections.

The antibody is directed against an antigen of interest. Preferably, the antigen is a biologically important polypeptide and administration of the antibody to a mammal may prevent or treat a disorder. However, antibodies directed against nonpolypeptide antigens (such as tumor-associated glycolipid antigens; see U.S. Pat. No. 5,091,178) are also contemplated.

Where the antigen is a polypeptide, it may be a transmembrane molecule (e.g. receptor) or ligand such as a growth factor. Exemplary antigens include molecules such as renin; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VIIIC, factor IX, tissue factor, and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin such as human serum albumin; Muellerian-inhibiting substance; relaxin A-chain; relaxin B-chain; prorelaxin; mouse gonadotropin-associated peptide; a microbial protein, such as beta-lactamase; DNase; IgE; a cytotoxic T-lymphocyte associated antigen (CTLA), such as CTLA-4; inhibin; activin; vascular endothelial growth factor (VEGF); receptors for hormones or growth factors; protein A or D; rheumatoid factors; a neurotrophic factor such as bone-derived neurotrophic factor (BDNF), neurotrophin-3, -4, -5, or -6 (NT-3, NT4, NT-5, or NT-6), or a nerve growth factor such as NGF-β; platelet-derived growth factor (PDGF); fibroblast growth factor such as aFGF and bFGF; epidermal growth factor (EGF); transforming growth factor (TGF) such as TGF-α and TGF-β, including TGF-β₁, TGF-β₂, TGF-β₃, TGF-β₄, or TGF-β₅; insulin-like growth factor-I and -II (IGF-I and IGF-II); des (1-3)-IGF-I (brain IGF-I), insulin-like growth factor binding proteins; CD proteins such as CD3, CD4, CD8, CD19 and CD20; erythropoietin; osteoinductive factors; immunotoxins; a bone morphogenetic protein (BMP); an interferon such as interferon-α, -β, and -γ; colony stimulating factors (CSFs), e.g., M-CSF, GM-CSF, and G-CSF; interleukins (ILs), e.g., IL-1 to IL-12; receptors to interleukins IL-1 to IL-12; selectins such as L, E, and P-selectin; superoxide dismutase; T-cell receptors; surface membrane proteins; decay accelerating factor; viral antigen such as, for example, a portion of the AIDS envelope; transport proteins; homing receptors; addressing; regulatory proteins; integrins such as CD11a, CD11b, CD11c, CD18, an ICAM, VLA-4 and VCAM; a tumor associated antigen such as HER2, HER3 or HER4 receptor; and fragments of any of the above-listed polypeptides.

When using recombinant techniques, the antibody can be produced intracellularly, in the periplasmic space, or directly secreted into the medium. If the antibody is produced intracellularly, as a first step, the particulate debris, either host cells or lysed cells, is removed, for example, by centrifugation or ultrafiltration. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human Υ₁, Υ₂, or Υ₄ heavy chains (Lindmark et al., J Immunol. Meth. 62:1-13 (1983)). Protein G is recommended for all mouse isotypes and for human Υ₃ (Guss et al., EMBO J. 5:1567-1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH₃ domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSET™ chromatography on an anion or cation exchange resin (such as a polyaspartic acid column), chromatofocusing, SDS-PAGE, and ammonium sulfate precipitation are also available depending on the antibody to be recovered.

Antibodies encompassed by the present invention include any antibodies, such as polyclonal, monoclonal, humanized antibodies. Preferred antibodies are IgG antibodies, such as IgG1, IgG2, IgG3, and IgG4. The present invention is exemplified with an IgG2 antibody, Visilizumab (Nuvion®), which is a humanized IgG2 monoclonal antibody targeting the CD3 antigen present on all T cells. T cells are an important component of the immune system and are normally present in the blood and bone marrow. Antibodies are proteins normally produced by our bodies to help the immune system fight off foreign substances. Visilizumab binds to the surface of activated T cells and causes their removal from the circulation. These cells are involved in inflammatory processes. Protein Design Labs, Inc. is developing nuvion as an immunosuppressive drug for treatment of GVHD (Graft-versus-host disease) and ulcerative colitis.

IV. Preparation of the Formulation

After the antibody of interest is prepared as described above, a pharmaceutical formulation comprising the antibody and one or more preservatives is prepared. The preservative(s) must not interfere with the physical, chemical, or biological activity of the antibody. For example, the preservative(s) must not cause antibody to aggregate or lose its activity. In addition, the preservative(s) must have efficacy to inhibit the growth of microorganisms, for example, bacteria or fungi, in the formulation.

The effects of antimicrobial parenteral preservatives (benzyl alcohol, chlorobutanol, methyl paraben, propyl paraben, phenol, and m-cresol) on antibody stability are assessed using size-exclusion chromatography (SEC), differential scanning calorimetry (DSC), right angle light-scattering, and ultraviolet (UV) spectroscopy. The antibody potency is tested by a cell-based fluorescence-activated cell sorting (FACS) method. Combinations of preservatives were examined using an I-optimal experimental design (Statistics for Experimenters: An Introduction to Design, Data Analysis, and Model Building, Box, George E. P. et al., John Wiley and Sons, Inc., 1978).

The antibody formulation of this invention comprises one or more preservatives that effectively inhibit microbial growth without affecting the antibody stability over time. The composition is a pharmaceutically acceptable liquid antibody formulation comprises an antibody and a single preservative benzyl alcohol at 0.5-0.75% (w/v). Applicants have found that benzyl alcohol at high concentration such as 1.0% causes antibody precipitation. At 0.5-0.75% (w/v) concentration, benzyl alcohol in general does not affect antibody activity or stability and is effective in inhibiting microorganism growth.

In some antibody formulations where 0.5-0.75% of benzyl alcohol would cause antibody to precipitate, a combination of preservatives is desirable. The combination of 0.15-0.2% (w/v) chlorobutanol and 0.3-0.5% (w/v) benzyl alcohol is effective in inhibiting microorganism growth in the antibody formulation without compromising the antibody activity or stability. For examples, a multidose antibody formulation contains an antibody, 0.35% benzyl alcohol and 0.2% chlorobutanol. Neither preservative alone is effective as an anti-microbial agent.

Another effective preservative combination is 0.1-0.2% (w/v) chlorobutanol and 0.05-0.1% (w/v) methyl paraben, which is effective on inhibiting microorganism growth in the antibody formulation without compromising the antibody stability or activity. For examples, a multidose antibody formulation contains an antibody, 0.1-0.2% (w/v) chlorobutanol and 0.1% (w/v) methyl paraben, or an antibody, 0.2% (w/v) chlorobutanol and 0.05-0.1% (w/v) methyl paraben. Neither preservative alone is effective as an anti-microbial agent.

The multidose antibody formulation comprises an antibody at any concentration suitable for its particular use. For example, a suitable antibody concentration can be 0.01-100 mg/mL, or 0.1-10 mg/mL.

The multidose antibody formulation can be any pH suitable for its particular use. For example, a suitable pH range can be pH 4-9, or 5.5-6.5, or 6.5-7.5. Buffers that control the pH in a suitable pH range can be used.

A surfactant is optionally included in the antibody formulation. Exemplary surfactants include nonionic surfactants such as polysorbates (e.g. polysorbates 20, 80, such as Tween® 20, Tween® 80) or poloxamers (e.g. poloxamer 188). The amount of surfactant added is such that it reduces aggregation of the formulated antibody and/or minimizes the formation of particulates in the formulation and/or reduces adsorption. The surfactant present in the formulation is in an amount from about 0.005% to about 0.5%, preferably from about 0.01% to about 0.1%, and more preferably from about 0.01% to about 0.05%.

A tonicity modifier, which contributes to the isotonicity of the formulations, is preferably included in the present composition. The tonicity modifier useful for the present invention includes salts and amino acids. Salts that are pharmaceutically acceptable and suitable for this invention include sodium chloride, sodium succinate, sodium sulfate, potassuim chloride, magnesium chloride, magnesium sulfate, and calcium chloride. Amino acids that are pharmaceutically acceptable and suitable for this invention include proline, alanine, L-arginine, asparagine, L-aspartic acid, glycine, serine, lysine, and histidine.

EDTA, which is commonly used to stabilize a protein formulation, is optionally included in the formulation. EDTA, as a chelating agent, may inhibit the metal-catalyzed oxidation of the sulfhydryl groups, thus reducing the formation of disulfide-linked aggregates.

The liquid antibody formulation of this invention is suitable for multidose administration because it is protected from microbial contamination during multiple dosage withdrawals from the vials.

The invention is illustrated further by the following examples, which are not to be construed as limiting the invention in scope of the specific procedures described in them.

### EXAMPLES

### Example 1

### Compatibility of Six Preservatives with Anti-CD3 Antibody

In this study, six preservatives were evaluated for compatibility with the antibody formulation (10 mg/mL antibody in a histidine buffer, pH 6.0, containing Tween® 80 and NaCl) and for antimicrobial efficacy. The preservatives' efficacy against various microbes was screened using a modified USP/EP preservative efficacy test to reduce cost and experiment time. After a preliminary screening of preservatives, an I-optimal experimental design approach was taken to identify the optimum preservative concentrations. The work reported here might be useful when developing multidose formulations for other biological products.

Materials

The humanized anti-CD3 monoclonal antibody (Visilizumab) was produced at Protein Design Labs Inc. (Fremont, Calif.). This study was conducted with 10 mg/mL protein, formulated in histidine buffer at pH 6.0, with Tween 80 and NaCl.

The preservatives benzyl alcohol, m-cresol, and phenol were obtained from Sigma (St. Louis, Mo.), and chlorobutanol, methyl paraben, and propyl paraben were obtained from U.S.P.C. Inc. (Rockville, Md.).

NOTATIONS/ABBREVIATIONS

| | |
|---|---|
| BA | benzyl alcohol |
| BP | British Pharmacopoeia |
| CB | chlorobutanol |
| DSC | differential scanning calorimetry |
| EDTA | ethylenediaminetetraacetic acid |
| EP | European Pharmacopoeia |
| FACS | fluorescence-activated cell sorting |
| LR | log reduction |
| MP | methyl paraben |
| PET | preservative efficacy test |
| PP | propyl paraben |
| SEC | size-exclusion chromatography |
| SN | sample number |
| Tₘ | denaturation temperature |
| TNTC | too numerous to count |
| USP | United States Pharmacopoeia |
| UV | ultraviolet |

Methods

Effects on Protein Stability

The compatibility of six parenteral preservatives (benzyl alcohol, chlorobutanol, methyl paraben, propyl paraben, phenol, and m-cresol) with the formulated humanized monoclonal antibody was tested. The preservatives were added to the formulated antibody based on their commonly used concentration ranges in marketed multidose products (3). Protein aggregation was suspected to be the primary degradation pathway. Preliminary evaluation of the preservatives was done initially by DSC and after 2 days of incubation at 50° C. by visual inspection for appearance and by SEC for soluble aggregates.

Because of the results seen in the preliminary evaluation, additional analyses were done using lower concentrations of the preservatives. Samples were incubated at 5° C. and 45° C. for 1 week, then analyzed with SEC, fluorescence spectroscopy, UV spectroscopy, and potency testing with a fluorescence-activated cell sorting (FACS) binding assay.

Size-Exclusion Chromatography (SEC)

The monomer content, soluble aggregates, and clips due to hydrolysis were monitored by size-exclusion chromatography. The analytical system employed consisted of an HPLC pump (Perkin Elmer, Series 410) and an autosampler (Perkin Elmer, ISS 2000) connected to a diode array detector (Perkin Elmer, 235C). Two size-exclusion chromatography columns (Tosohaas TSK-Gel, G3000SW_{XL},), connected in tandem, were used for sample separation. The composition of the mobile phase was 200 mM KPO₄, 150 mM NaCl, pH 6.9. Samples were diluted to 1 mg/mL and a sample volume of 40 μL was injected for analysis. The flow rate was 1.0 mL/min, and detection was at 220 and 280 nm.

Differential Scanning Calorimetry (DSC)

A decrease in the denaturation temperature reflects a destabilizing effect of the preservative on formulation stability. The denaturation temperature (Tₘ) of the sample was measured using the Pyris 1 differential scanning calorimeter (Perkin Elmer). A sample volume of 50 μL was taken from a 10 mg/mL protein sample and sealed in a stainless steel pan. The sample was held at 32° C. for 2 minutes, and heated to 100° C. at the rate of 10° C./min.

Potency Testing

The biological activity (potency) of the protein was measured by flow cytometry, based on the binding of the antibody antigen expressed on human T cells.

Preservative Screening Test (Bactericidal/Fungicidal Activity)

The efficacy of the preservative against various microorganisms was measured using a modified USP/EP preservative efficacy test. These tests were conducted at Microconsult Inc. (Dallas, Tex.). In the procedure, formulations were tested against the following microorganisms: Escherichia coli, Staphylococcus aureus, Pseudomonas aeruginosa, Candida albicans, and Aspergillus niger. The three bacterial strains were inoculated together at a total concentration of ˜10⁵ cfu/mL, and the two fungi were inoculated together at a total concentration of ˜10⁵ cfu/mL. Samples were incubated for 7 days at room temperature (25° C.), and the total bacterial and fungal counts were measured using a colony counter. The log reduction (LR) values for the bacterial and fungal counts were calculated as log (initial count/final count).

In the unmodified USP/EP preservative efficacy tests, each microorganism is tested separately at a concentration of ˜10⁵ cfu/mL. The USP and EP regulatory guidelines are listed in Table I; note that the EP guidelines are more stringent than those of the USP, and that the EP guidelines offer a minimal level that must be achieved (B criteria) and a suggested level that is recommended (A criteria). The preservative efficacy test was modified in this study to reduce the total sample requirement and cost per analysis. Although the bacterial and fungal strains were not tested individually at specified concentrations, by comparing the overall bacterial and fungal log reduction values with the regulatory requirements, an assessment is made of the efficacy of the preservative against these microorganisms.

TABLE I
USP 24 and EP 2 requirements for preservatives efficacy testing

EP 2 Requirements

| | | | |
|---|---|---|---|
| | USP 24 | Suggested | Minimum |
| Time point | Requirements | (A Criteria) | (B Criteria) |

Requirements for Bacterial Log Reduction

| | | | |
|---|---|---|---|
| 6 hours | Not required | 3 | Not required |
| 24 hours | Not required | No recovery | 1 |
| 2 days | Not required | No recovery | Not required |
| 7 days | 1 | No recovery | 3 |
| 14 days | 3 | No recovery | Not required |
| 21 days | No increase | No recovery | Not required |
| 28 days | No increase | No recovery | No increase |

Requirements for Fungal Log Reduction

| | | | |
|---|---|---|---|
| 7 days | Not required | 2 | Not required |
| 14 days | No increase | No increase | 1 |
| 28 days | No increase | No increase | No increase |

SP = United States Pharmacopoeia
EP = European Pharmacopoeia

I-Optimal Experimental Design

An I-optimal experimental design was used to evaluate and model the effects of single and combined preservatives on formulation stability and antimicrobial efficacy. Because the preservatives were compatible with the formulation only at low concentrations, we were especially interested in evaluating whether combinations of preservatives enhanced the antimicrobial efficacy of the formulation. The preservatives benzyl alcohol, chlorobutanol, methyl paraben, and propyl paraben were examined in the concentration ranges of 0-0.75%, 0-0.2%, 0-0.1%, and 0-0.01%, respectively.

The formulations were prepared by adding the preservatives at the desired concentrations as per the I-optimal design table (Table II), generated using the software Strategy (Experiment Strategies Foundation & Process Builder, Inc.). All samples were incubated at 37° C. for 9 weeks. Because protein aggregation was known to be the primary degradation pathway, protein stability was examined by SEC and right-angle light scattering to monitor the formation of soluble and insoluble aggregates, respectively, and by UV spectroscopy to monitor changes in the protein concentration. The biological activity of the samples was assessed after 1 month at 37° C. Furthermore, to assess the antimicrobial efficacy of the formulations, samples were examined by the preservative screening test at the initial time point. Samples were incubated at room temperature, and the aerobic plate counts were measured based on the minimum requirements of the USP and BP/EP preservative efficacy tests at 24 hours, 7 days, and 14 days.

TABLE II
I-Optimal Experimental Design: Comparison of measured and predicted log
reduction (LR) values for bacteria and fungi

| | Preservative | | | | | | | |
|---|---|---|---|---|---|---|---|---|
| | concentration (%) | | | | Log Reduction Values | | | |
| | BA | CB | MP | PP | Bacteria | | Fungi | |
| S.N.^{a} | % | % | % | % | Measured | Predicted^{b} | Measured | Predicted^{c} |
| 1(2) | 0.33 | 0.10 | 0.05 | 0.005 | 2.11 | 2.90 ± 0.10 | 3.72 | 3.69 |
| 2 | 0.00 | 0.20 | 0.03 | 0.000 | 2.36 | 2.32 ± 0.19 | 1.75 | 1.75 |
| 3 | 0.00 | 0.05 | 0.10 | 0.000 | 2.59 | 2.58 ± 0.19 | 3.72 | 3.72 |
| 4 | 0.75 | 0.10 | 0.05 | 0.005 | 3.83 | 3.40 ± 0.14 | 3.72 | 3.80 |
| 5 | 0.47 | 0.20 | 0.05 | 0.010 | 3.83 | 3.67 ± 0.18 | 3.72 | 3.75 |
| 6 | 0.47 | 0.10 | 0.00 | 0.000 | 3.83 | 3.67 ± 0.10 | 3.72 | 3.75 |
| 7 | 0.66 | 0.20 | 0.10 | 0.000 | 3.83 | 3.84 ± 0.19 | 3.24 | 3.23 |
| 8 | 0.00 | 0.00 | 0.07 | 0.010 | 1.54 | 1.53 ± 0.19 | 3.72 | 3.72 |
| 9 | 0.33 | 0.10 | 0.05 | 0.005 | 3.83 | 2.90 ± 0.10 | 3.72 | 3.70 |
| 10 | 0.00 | 0.15 | 0.00 | 0.010 | 0 | −0.01 ± 0.19 | 2.72 | 2.72 |
| 11 | 0.47 | 0.00 | 0.10 | 0.005 | 3.83 | 3.67 ± 0.18 | 3.72 | 3.75 |
| 12(3) | 0.66 | 0.00 | 0.00 | 0.010 | 3.83 | 3.83 ± 0.11 | 3.72 | 3.72 |
| 13 | 0.75 | 0.20 | 0.00 | 0.005 | 3.49 | 3.52 ± 0.18 | 3.72 | 3.69 |
| 14 | 0.00 | 0.20 | 0.10 | 0.007 | 3.83 | 3.81 ± 0.19 | 3.72 | 3.72 |
| 15(2) | 0.75 | 0.00 | 0.05 | 0.000 | 3.83 | 3.91 ± 0.13 | 3.72 | 3.70 |
| 16(2) | 0.00 | 0.00 | 0.00 | 0.003 | 0 | −0.01 ± 0.13 | 0.00 | 0.00 |
| 17 | 0.75 | 0.10 | 0.10 | 0.010 | 3.83 | 4.00 ± 0.18 | 3.72 | 3.69 |

^{a}S.N. = sample number; Numbers in parenthesis indicate replicates included in the design. In those cases, mean values are given.
^{b}Response variation is reported based on 95% confidence limits
^{c}Pooled standard deviation for this data set was zero
BA = benzyl alcohol
CB = chlorobutanol
MP = methyl paraben
PP = propyl paraben

In the I-optimal model, the main effect and the interaction effects of various factors are determined by fitting the data to a second-order quadratic equation:

$$Y = {b}_{o} + {\sum }_{i = 1}^{k} {b}_{i} {x}_{i} + {\sum }_{i = 1}^{k} {b}_{ii} {x}_{i}^{2} + {\sum }_{i < j} \sum {b}_{ij} {x}_{i} {x}_{j} \qquad ( 1 )$$

where Y is the dependent variable or the measured response, and xᵢ represents the independent variable that corresponds to the concentration of excipient i. The model coefficients determined by regression analysis define the response surface; bₒ is a constant term, bᵢ indicates the main effect of excipient xᵢ, and bᵢⱼ represents the interaction effect between excipients i and j. These model coefficients were used to generate response surfaces that simulate the effect of preservatives on the desired response.

Results

Effects on Antibody Stability

The results from the preliminary testing after 2 days of incubation at 50° C. indicated that the preservative concentration affected antibody stability (Table III). Benzyl alcohol caused sample precipitation at concentrations ≧2%. At a concentration of 1.0%, the sample was slightly cloudy, and the monomer content was only ˜13%. However, at 0.5% benzyl alcohol, the sample was clear, and the monomer content was ˜92%. The loss in monomer content in the 1.0% sample was primarily due to the formation of soluble aggregates. Previously, the destabilizing effect of benzyl alcohol on recombinant human interferon gamma has been reported to be due to the disruption of the protein's tertiary structure, making it more susceptible to aggregation (10).

In the presence of chlorobutanol, the formulations were clear, however, as with benzyl alcohol, the protein formed soluble aggregates at higher preservative concentrations. The monomer content values in samples containing 0.5% and 0.1% chlorobutanol were 3.3% and 97%, respectively.

The antibody was stable in the presence of methyl and propyl paraben. Despite being tested at their highest recommended concentrations (0.1% for methyl paraben and 0.02% for propyl paraben), the monomer content values were ˜91% and 85%, respectively.

When tested under identical conditions, the control formulation containing no preservatives showed 97.4% monomer.

Phenol and m-cresol considerably destabilized the protein; m-cresol precipitated the protein, while phenol caused the formation of both soluble and insoluble aggregates. Thus, these two preservatives were not evaluated further in this study.

The denaturation temperature of the control formulation was ˜80° C. In the presence of benzyl alcohol, the denaturation temperature was considerably reduced; for formulations containing 2.0% and 0.5% benzyl alcohol, the measured Tₘ values were 72.3 and 78.5° C., respectively. Similarly, at 0.5% chlorobutanol, the Tₘ dropped by 1.5° C. relative to the control formulation. The addition of methyl and propyl paraben, however, did not affect the denaturation temperature. These results are thus consistent with the SEC results, indicating the compatibility of the parabens with the formulated protein and the instability of the protein at higher concentrations of benzyl alcohol and chlorobutanol.

TABLE III
Preliminary screening of preservatives to evaluate compatibility with the
formulated protein. SEC and visual analysis were done after 2 days of
incubation at 50° C. DSC analysis was conducted at the initial time point,
t₀.

| | | | Percent | |
|---|---|---|---|---|
| | Concentration | | monomer | Tₘ (° C.) |
| Preservative | (%) | Visual analysis | (by SEC) | (by DSC) |
| Benzyl alcohol | 2 | Precipitated | nd | 72.3 |
| | 1.0 | Slightly cloudy | 13.4 | 75.8 |
| | 0.5 | Clear | 92.3 | 78.5 |
| Chlorobutanol | 0.5 | Clear | 3.3 | 78.5 |
| | 0.2 | Clear | nd | 79.9 |
| | 0.1 | Clear | 96.9 | nd |
| Methyl paraben | 0.1 | Clear | 91.3 | nd |
| | 0.05 | Clear | nd | 80.4 |
| Propyl paraben | 0.02 | Clear | 85.3 | 80.6 |
| Phenol | 0.5 | Cloudy | 20.0 | nd |
| | 0.1 | Slightly cloudy | 62.7 | nd |
| m-cresol | 0.3 | Precipitated | nd | nd |
| | 0.1 | Precipitated | nd | nd |
| Control | 0.0 | — | 97.4 | 80.1 |

nd = not determined

Based on the results obtained using preservatives at typical commercial concentrations in the preliminary investigation, preservative concentrations were reduced in further analyses. Results from samples containing lower concentrations of preservatives stored for 7 days at 5° C. and 45° C. showed that samples could not be distinguished based on fluorescence and UV spectroscopy measurements, relative to the control formulation (data not shown). These spectra represent microenvironments of the protein around the aromatic residues and would indicate a red shift if the environment becomes more hydrophobic due to protein unfolding. The SEC (% monomer) and bioactivity (% potency) results are shown in Table IV. SEC results indicate that the stability of all formulations correlated with the temperature; the monomer content for all samples was >98% at 5° C. (data not shown), while it varied from 88.0% to 98.3% at 45° C. In particular, samples formulated with benzyl alcohol were unstable due to the formation of soluble aggregates.

TABLE IV
Effect of preservatives on protein stability and antimicrobial Efficacy. Samples
were incubated for 1 week at the Indicated Temperatures.

| | | Monomer | Potency | Bacterial × 10⁴ | Fungi × 10⁵ |
|---|---|---|---|---|---|
| | | (%) | (%) | (cfu/mL)^{a} | (cfu/mL)^{b} |
| | Concentration | Storage temperature | | | |
| Preservative | (%) | 45 ° C. | 45 ° C. | 25 ° C. | 25 ° C. |
| Benzyl alcohol | 0.75 | 87.97 | 74 | 0 | 0 |
| | 0.5 | 93.92 | 75 | 0 | 0 |
| | 0.1 | 97.65 | 81 | TNTC | 12 |
| Chlorobutanol | 0.2 | 96.75 | 63 | 46 | 0 |
| | 0.1 | 97.52 | 67 | TNTC | 14 |
| | 0.05 | 97.62 | 60 | TNTC | 18 |
| Methyl | 0.1 | 97.18 | 75 | 56 | 0 |
| paraben | 0.05 | 97.43 | 75 | TNTC | 2 |
| | 0.01 | 98.31 | 73 | TNTC | 26 |
| Propyl paraben | 0.01 | 97.34 | 68 | TNTC | 0 |
| | 0.0075 | 97.89 | 74 | TNTC | 2 |
| Control | — | 97.48 | 69 | TNTC | 20 |

^{a}Total bacterial count = 8.78 × 10⁴ cfu/mL
^{b}Total fungal count = 2.84 × 10⁵ cfu/mL
TNTC = Too numerous to count

Potency was measured only in samples incubated at 45° C. Based on the inherent variability of this assay (standard deviation˜8%), the potency of the preservative-containing formulations was equivalent to that of the control formulation. Our earlier studies have shown that at high temperatures, oxidation of methionine residues in the Fc region is catalyzed by the histidine buffer, causing structural changes in the protein and a loss in biological activity. However, at ambient temperature, the process slows down considerably and the molecule satisfactorily retains its biological activity for the target shelf life. Thus, the data here indicate that under the examined conditions, the loss in biological activity at 45° C. is not catalyzed by the preservatives in the formulation.

Preservative Screening Test

Results of the preservative efficacy tests showed that the formulations containing 0.75% and 0.5% benzyl alcohol are potential candidates to meet the USP/EP criteria (Table II). Both formulations demonstrated a complete kill of the tested bacterial and fungal species after 7 days. For all other samples, the total bacterial count after 7 days was either too numerous to count (TNTC), or no effective reduction in the bacterial count was observed. The antimicrobial efficacy was also satisfactory against fungi for formulations containing at least 0.5% benzyl alcohol, and for formulations containing parabens and chlorobutanol at their highest concentration. In the stability testing reported above, the stability of the protein strongly correlated with the concentration of benzyl alcohol. Therefore, it is desirable to find combined preservatives at lower concentrations, which attain the desired antimicrobial efficacy without affecting protein stability.

I-Optimal Analyses

The SEC, right angle light-scattering, and UV spectroscopy responses after 9 weeks of incubation at 37° C., and the bioactivity response after 1 month at 37° C. were modeled using the I-optimal design (data not shown). The regression results for the responses were not statistically significant. The samples could not be statistically distinguished from the control formulation, and thus, the stability of the samples was not adversely affected by combined preservatives in the examined concentration range. However, the formulations having combined preservatives differed markedly in their antimicrobial efficacy compared with formations having a single preservative.

The reduction in the bacterial and fungal counts following 14 days of incubation at room temperature was taken as the measured response and modeled using the I-optimal design. The data at the 24-hour and 7-day time points also followed a similar trend. The regression yielded a set of coefficients that correlates the concentration of the preservatives to the log reduction in the bacterial and fungal counts (see Equation 1). Thus, the response in the region of interest can be simulated for optimizing the formulation components. Excellent agreement was observed between the experimentally measured and model-predicted responses (Table II), confirming a good fit between the model and the experimental data.

The b-coefficients determined by regression analysis are listed in Table V. Benzyl alcohol, chlorobutanol, and methyl paraben showed statistically significant antimicrobial efficacy against bacteria, the effect being strongest for benzyl alcohol, followed by methyl paraben and chlorobutanol. Propyl paraben, on the other hand, was not effective against the tested bacterial strains. Interaction effects were also statistically significant between various preservatives; the strongest positive interaction (synergistic effect) was between methyl paraben and propyl paraben, and the strongest negative interaction was between benzyl alcohol and methyl paraben. Other positive interaction effects included benzyl alcohol and propyl paraben, and chlorobutanol and methyl paraben. The b-coefficients for fungi were also statistically significant. All selected preservatives had a positive antifungal efficacy, the strongest effect being observed for benzyl alcohol followed by methyl paraben. All interaction effects were, however, negative.

TABLE V
I-Optimal Design: Computed b-coefficients for bacteria and fungi

b-coefficients

| Sample | Bacterial LR^{a} | Fungal LR^{b} |
|---|---|---|
| Constant | 3.04 ± 0.10 | 3.75 |
| BA | 1.04 ± 0.05 | 0.51 |
| MP | 0.60 ± 0.05 | 0.46 |
| CB | 0.27 ± 0.05 | 0.07 |
| PP | −0.14 ± 0.05 | 0.29 |
| BA-CB | −0.45 ± 0.07 | −0.20 |
| BA-MP | −0.73 ± 0.07 | −0.82 |
| BA-PP | 0.16 ± 0.07 | −0.37 |
| CB-MP | 0.12 ± 0.07 | −0.30 |
| CB-PP | −0.07 ± 0.07 | −0.08 |
| MP-PP | 0.35 ± 0.07 | −0.06 |
| BA-BA | −0.68 ± 0.11 | −0.46 |
| CB-CB | 0.17 ± 0.10 | −0.64 |
| MP-MP | 0.12 ± 0.10 | 0.00 |
| PP-PP | 0.26 ± 0.10 | 0.40 |

^{a}Response variation is reported based on 95% confidence limits.
^{b}Pooled standard deviation for the data was zero.
LR = log reduction value
BA = benzyl alcohol
CB = chlorobutanol
MP = methyl paraben
PP = propyl paraben

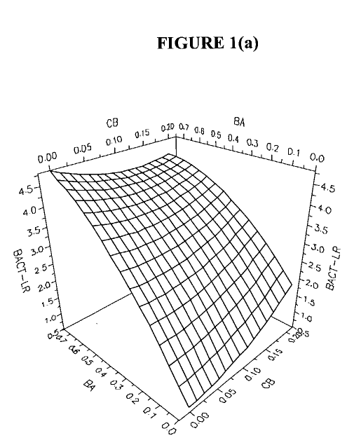

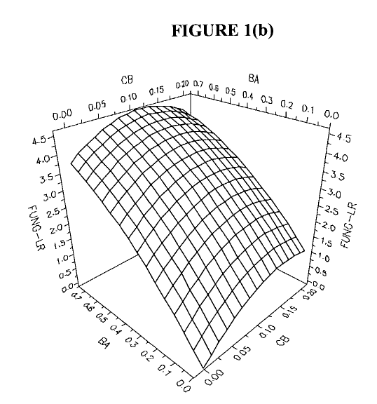

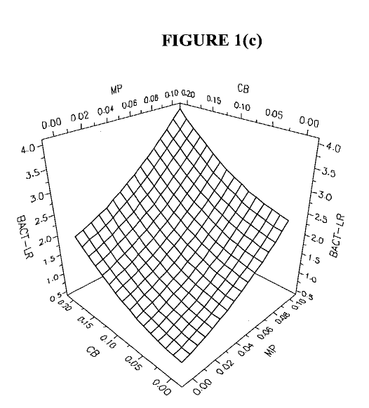

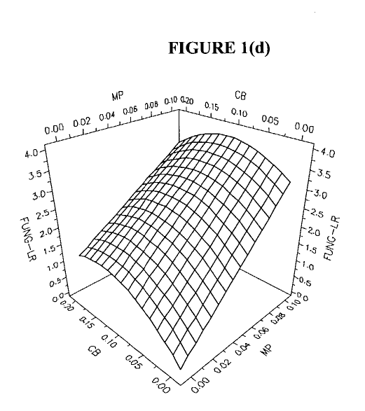

The efficacy of the single and combined preservatives was evaluated by comparing the log-reduction values predicted by the model with the regulatory requirements. FIGS. 1a and 1b show the effects of benzyl alcohol and chlorobutanol on the log reduction of the bacterial and fungal counts, respectively. The antimicrobial efficacy against bacteria and fungi increased with increasing concentrations of benzyl alcohol and chlorobutanol, however it is unlikely that chlorobutanol alone can provide adequate protection against bacteria or fungi. The simulations predict that as single preservatives, 0.75% benzyl alcohol and 0.2% chlorobutanol (their maximal concentrations) would provide log reduction values of 4.8 and 2.0, respectively, for bacteria and of 3.7 and 1.2, respectively, for fungi. These results indicate that benzyl alcohol is effective in preserving the formulation against both bacteria and fungi.

The results also show that combinations of benzyl alcohol and chlorobutanol do not enhance the antimicrobial efficacy against bacteria, however they do enhance the antimicrobial efficacy against fungi. For example, by using 0.75% benzyl alcohol and 0.125% chlorobutanol, the log-reduction in the fungal count can be increased from 3.7 to 4.6. However, the bacterial log reduction under these conditions drops from 4.8 to 4.3. These model predictions can also be advantageous in seeking alternatives if protein stability, preservative toxicity, or other factors require the preservative to be in a specific concentration range.

Combining chlorobutanol and methyl paraben has a synergistic effect on antimicrobial activity against bacteria and fungi (FIGS. 1c and 1d, respectively. The model simulations indicated maximal log reductions of 2.0 and 2.3 for the individual preservatives against bacteria; their combination resulted in a significant improvement of up to 4.0 log reductions. The log-reduction in the fungal count increased marginally from 3.2 to 3.9. Thus, the combination of chlorobutanol and methyl paraben may offer a promising alternative to the use of benzyl alcohol.

Based on these results, to evaluate the efficacy of the preservative screening approach undertaken in this study, the protein was formulated with 0.75% benzyl alcohol, and its stability and bioactivity were monitored over time (data not shown). The antimicrobial efficacy of the preservative was monitored by the USP and EP/BP preservative efficacy tests. Results indicated that in samples containing 0.75% benzyl alcohol, protein stability was comparable to that of the control formulation, and the USP and EP/BP (criterion B only) regulatory requirements were satisfied.

Conclusions

The effects of five preservatives (benzyl alcohol, chlorobutanol, methyl paraben, propyl paraben, m-cresol, and phenol) on the stability of a humanized monoclonal antibody were examined in order to develop a multidose intravenous formulation. Preservatives were screened based on their effect on the physical stability of the formulations using various analytical techniques and on their antimicrobial efficacy using a modified preservative efficacy test. Antibody stability in the presence of the parabens and low concentrations of chlorobutanol compared well with that of the control formulation. Benzyl alcohol caused significant aggregation at high concentrations (≧1.0%), however, it was the most effective preservative in maintaining antimicrobial efficacy against bacteria and fungi. Phenol and m-cresol were not compatible with the protein and caused protein precipitation.

An I-optimal experimental design was used to monitor the individual effects of each preservative and to examine combinations of preservatives that result in a synergistic effect. Based on these results, as a single preservative, benzyl alcohol (0.5%-0.0.75% can meet the regulatory requirements. As combinations, benzyl alcohol-chlorobutanol and benzyl alcohol-methyl paraben enhanced the antimicrobial efficacy of the formulation against fungi, and chlorobutanol and methyl paraben enhanced the antimicrobial efficacy against both bacteria and fungi, at all concentrations of both preservatives.

The invention, and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the scope of the present invention as set forth in the claims. To particularly point out and distinctly claim the subject matter regarded as invention, the following claims conclude this specification.

### REFERENCES

1. P. McGoff and D. S. Scher. Solution formulation of proteins/peptides. In E. J. McNally (ed.), Protein Formulation and Delivery, Marcel Dekker Inc., New York, 2000, pp. 139-158.

2. M. J. Akers and M. R. Defelippis. Peptides and proteins as parenteral solutions. In S. Frokjaer and L. Hovgaard (eds.). Pharmaceutical Formulation Development of Peptides and Proteins, Taylor and Francis, Philadelphia, 2000, pp. 145-177.

3. J. A. Bontempo. Formulation development. In J. A. Bontempo (ed.), Development of Biopharmaceutical Parenteral Dosage Forms, Marcel Dekker Inc., New York, 1997, pp. 109-142.

4. L. Lachman, S. Weinstein, G. Hopkins, S. Slack, P. Eisman, and J. Cooper. Stability of antibacterial preservatives in parenteral solutions I. J Pharm Sci. 51(3):224-232 (1962).

5. K. Kakemi, H. Sezaki, E. Arakawa, and K. Ideda. Interaction of parabens and other pharmaceutical adjuvants with plastic containers. J Chem Pharm Bull. 19(12):2523-2529 (1971).

6. M. J. Akers. Considerations in selecting antimicrobial preservatives for parenteral product development. Pharm Technol. 8(5):36-44 (1984).

7. C. R. W. Beasley, P. Rafferty, and S. T. Holgate. Bronchoconstrictor properties of preservatives in ipratropium bromide (Atrovent) nebulizer solution. Br Med J. 294:1197-1198 (1987).

8. Y. F. Maa and C. C. Hsu. Aggregation of recombinant human growth hormone induced by phenolic compounds. Int J Pharm. 140:155-168 (1996).

9. Y. Kim, C. A. Rose, Y. Liu, Y. Ozaki, G. Datta, and A.T. Ut. FT-IR and near-infrared FT-Raman studies of the secondary structure of insulinotropin in the solid state: α-helix to β-sheet conversion by phenol and/or high shear force. J Pharm Sci. 83:1175-1180 (1994).

10. X. M. Lam, T. W. Patapoff, and T. H. Nguyen. The effect of benzyl alcohol on recombinant human interferon-γ. Pharm Res. 14:725-729 (1997).

What is claimed is:

1. A liquid pharmaceutical formulation comprising:
an IgG antibody,
0.15-0.2% (w/v) chlorobutanol, and
0.3-0.5% (w/v) benzyl alcohol,
wherein said antibody is stable in said formulation and said formulation is effective in inhibiting antimicrobial activity.

2. A liquid pharmaceutical formulation comprising:
an IgG antibody,
0.1-0.2% (w/v) chlorobutanol, and
0.05-0.1% (w/v) methyl paraben,
wherein said antibody is stable in said formulation and said formulation is effective in inhibiting antimicrobial activity.

3. The liquid pharmaceutical formulation according to claim 1 or 2, wherein said IgG antibody is an IgG 2 antibody.

4. The liquid pharmaceutical formulation according to claim 1 or 2, wherein said IgG antibody is a humanized antibody

5. The liquid pharmaceutical formulation according to claim 1 or 2, wherein said IgG antibody is a humanized anti-CD3 antibody.

6. The liquid pharmaceutical formulation according to claim 5, wherein said humanized anti-CD3 antibody is Visilizumab.

7. A liquid pharmaceutical formulation comprising:
a humanized anti-CD 3 antibody and 0.5-0.75% benzyl alcohol,
wherein said antibody is stable in said formulation and said formulation is effective in inhibiting antimicrobial activity.

8. The liquid pharmaceutical formulation according to claim 7, wherein said humanized anti-CD3 antibody is Visilizumab

9. The liquid formulation according to claim 1, 2, or 7, further comprising a tonicity modifier.
